(19) United States
(12) Patent Application Publication
(10) Pub. No.: US 2022/0160580 A1
(43) Pub. Date: May 26, 2022

(54) CONNECTOR FOR A BAG CONTAINING LIQUID SOLUTIONS FOR MEDICAL AND NUTRITIONAL USE

(71) Applicant: PAOLO GOBBI FRATTINI S.R.L., Milano (IT)

(72) Inventors: Paolo Giuseppe GOBBI FRATTINI, Sondalo SO (IT); Dario Caspani, Grosio SO (IT)

(73) Assignee: PAOLO GOBBI FRATTINI S.R.L., Milano (IT)

(21) Appl. No.: 17/531,841

(22) Filed: Nov. 22, 2021

(30) Foreign Application Priority Data
Nov. 23, 2020 (IT) 102020000028040

(51) Int. Cl. A61J 1/14; A61J 1/10

(52) U.S. Cl. CPC A61J 1/1406; A61J 1/1475; A61J 1/10

(57) ABSTRACT

A connector (3) which is applicable to a bag (1) for containing liquid solutions for medical or nutritional use comprises a coupling part (11) for coupling to an end of a small tube (2) projecting from the bag (1) and provided with an axial hole (5) closed by a pierceable inner membrane (6). The coupling part (11) includes two coaxial tubes (12, 13) which define an annular space (14) extending axially with a constant cross-section, into which said end of the small tube (2) is forcibly insertable by axial pressure.

### BACKGROUND OF THE INVENTION

The present invention relates to a connector for a bag for containing liquid solutions for medical or nutritional use, in particular a dispensing connector for a bag of the aforesaid type.

In hospitals, nursing homes, retirement homes, and so on, wide use is made of systems for dispensing liquid solutions of drugs or nutrients which, from initial containment bags, enable the liquid solutions to reach the patients' veins.

Dispensing usually takes place through a small tube which extends from the bag in a single piece with the bag itself and a dispensing connector normally closed by a pierceable membrane and having a frangible end part which, for use, is removed to allow the insertion of a piercing terminal placed at the inlet of an infusion set or other appropriate dispensing device.

An important problem relates to the coupling between the small tube and the connector, which is performed by inserting a corresponding tubular part of the connector into the end of the small tube by pressure.

Obviously, the coupling needs to be highly integral so as to prevent any disastrous detachments between the small tube and the connector and ensure a perfect liquid seal, with both properties being capable of lasting over time.

This prerogative is ensured if the sterilization of the bag with the related small tube and connector is performed hot in an autoclave, but this may not be the case with cold sterilization resulting in discomfort and elements of danger for patients and medical personnel.

US 2011/0022024 A1 describes a connector for infusion or transfusion bags which has features similar to those defined in the preamble of claim 1, and in particular it includes a coupling part with two coaxial tubes which define a truncated cone space intended to insert the free end of the small tube leaving the bag.

### SUMMARY OF THE INVENTION

In view of this state of the art, it has been the object of the present invention to provide a coupling between the small tube and the connector which gives increased integrity and liquid sealing capacities in any use situation and whatever sterilization technique is used.

It was another object to provide a coupling having the aforesaid properties which was usable for any type of connector, for both dispensing and filling the bag.

According to the present invention, such an object has been achieved by the connector defined in the claims.

The seal against the detachment between small tube and connector and against liquid leaks is thus apparently increased, the forced contact surface between the small tube and the coupling part of the connector being double and lengthened over the entire extension of the annular space with constant section.

Possibly, the sealing capacity can be further increased by associating roughness elements with the inner surface of the outer tube and the outer surface of the inner tube of the coupling part of the connector, which increase the integrity thereof with the tube.

### DESCRIPTION OF THE DRAWINGS

The features of the present invention will become more apparent from the following detailed description of a practical embodiment thereof, shown by way of non-limiting example in the accompanying drawings, in which:

FIG. 1 shows a liquid solution bag before coupling it to a dispensing connector according to the present invention;

FIG. 2 shows a front view of a connector according to the present invention;

FIG. 3 shows a side view of the same connector;

FIG. 4 shows said connector in an axial section according to line IV-IV in FIG. 2;

FIG. 5 shows said connector in an axial section according to line V-V in FIG. 3;

FIG. 6 shows said connector in a cross section according to line VI-VI in FIG. 5;

FIG. 7 shows the bag and the connector once the coupling has taken place;

FIG. 8 shows an axial section of the coupling obtained between the connector and the small dispensing tube of the bag;

FIG. 9 shows a cross-section of said coupling according to the line IX-IX in FIG. 8;

FIGS. 10-12 show the steps of applying an infusion set to the connector in the previous figures.

### DETAILED DESCRIPTION OF THE INVENTION

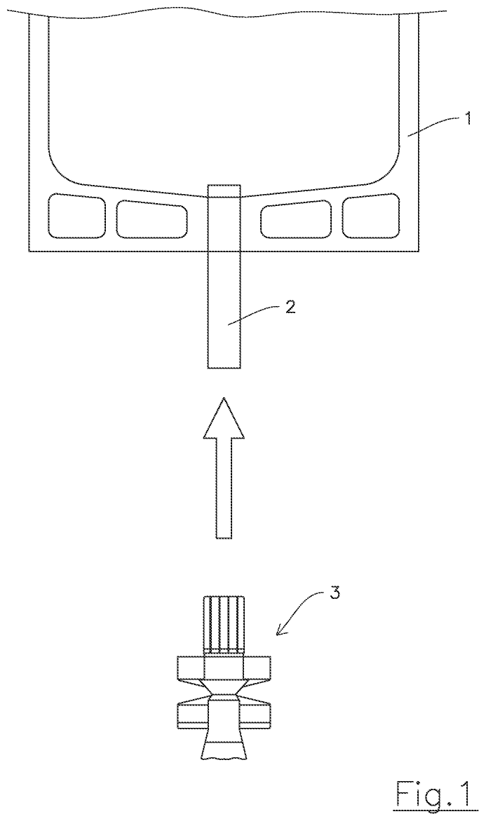

More in particular, FIG. 1 shows a flexible liquid solution bag 1 from which a small plastic tube 2 made in a single piece with the bag itself extends outwards.

Before or after filling the bag with a liquid solution, a connector 3 according to the present invention is coupled to the small tube 2, having the task of keeping the small tube itself closed until the time of dispensing the liquid solution by means of an infusion set or other dispensing device applied to the connector 3.

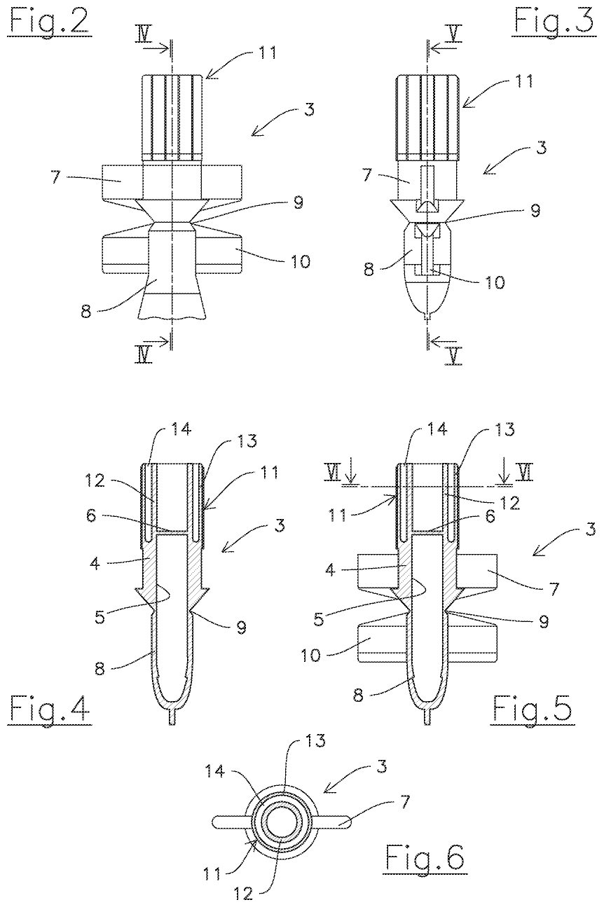

A connector 3 according to the present invention is shown in detail in FIGS. 2-6 and comprises a single body 4 formed by a coupling part 11 with an axial hole 5 normally closed by a pierceable inner membrane 6 and lateral gripping fins 7 and by a closing part 8 which is detachable from the coupling part 11 at a frangible weakened section 9 and in turn provided with lateral gripping fins 10. Both fins 7 and 10 are positioned close to the weakened section 9.

The coupling part 11, intended to receive the free end of the small tube 2 of bag 1, is formed by two coaxial tubes 12 and 13, inner and outer tubes, respectively, which define an annular space 14 which extends axially with a constant section. Advantageously, the outer surface of the inner tube 12 and the inner surface of the outer tube 13 can have various type of roughness elements, not shown in the drawings.

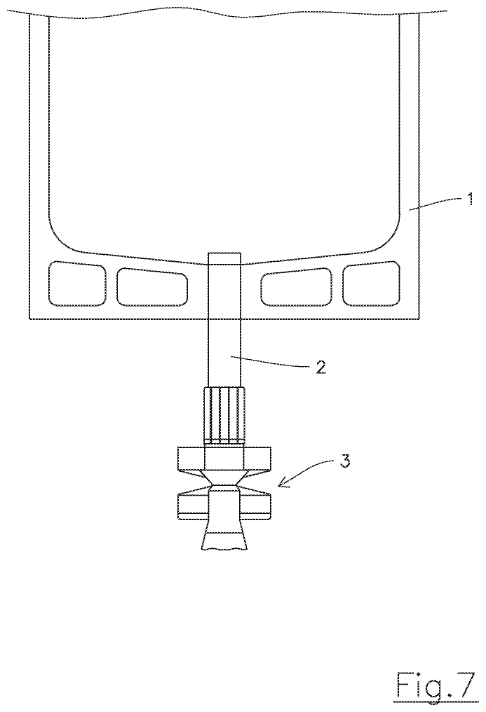

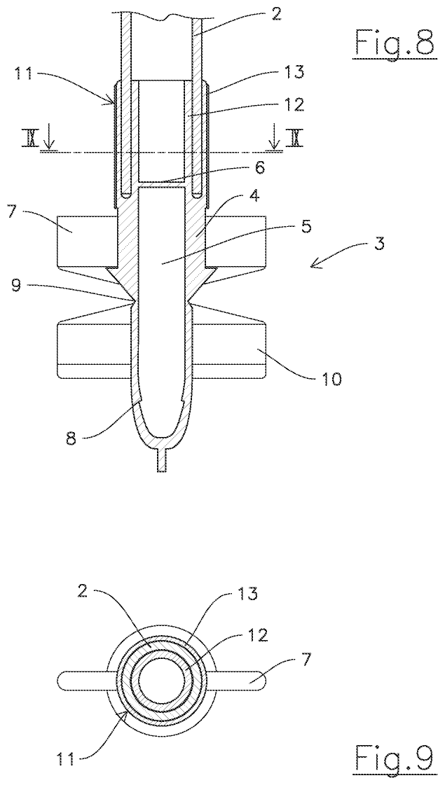

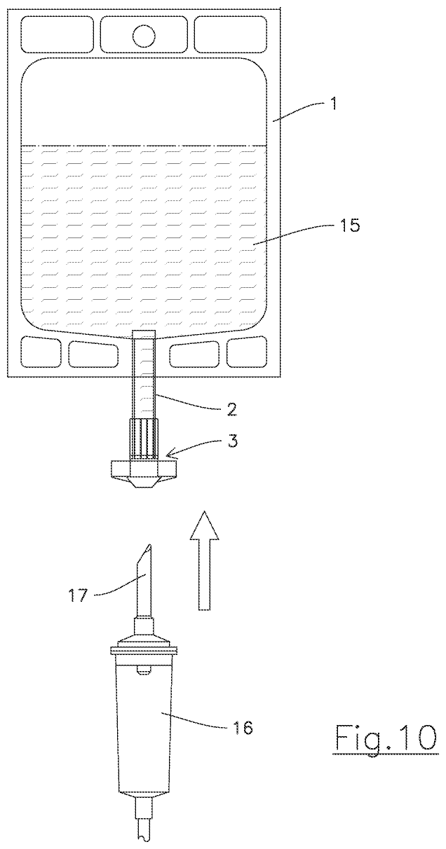

FIG. 7 shows the connector 3 coupled to the small dispensing tube 2 of bag 1. The coupling is obtained as shown in FIGS. 8 and 9, i.e. by inserting the outer end of the small tube 2 forcibly by axial pressure into the annular space 14 between the two coaxial tubes 12 and 13 of the tubular part 11. Any roughness elements provided for the facing surfaces of the two coaxial tubes can increase the integrity between the small tube 2 and the connector 3 and the stability of the coupling thereof.

A fluid solution dispensing device can be connected to the bag 1 filled with a fluid solution 15 and having the small dispensing tube 2 and the connector 3 coupled to each other, which device may be of various types, for example an infusion set 16 with an axially hollow piercing terminal 17, as shown in FIG. 10.

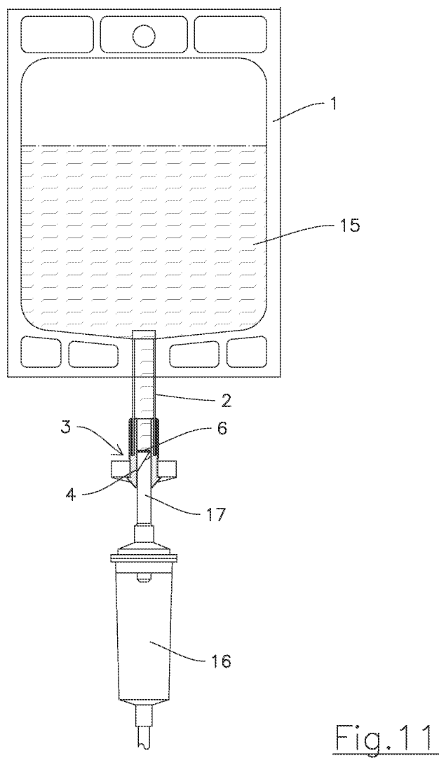

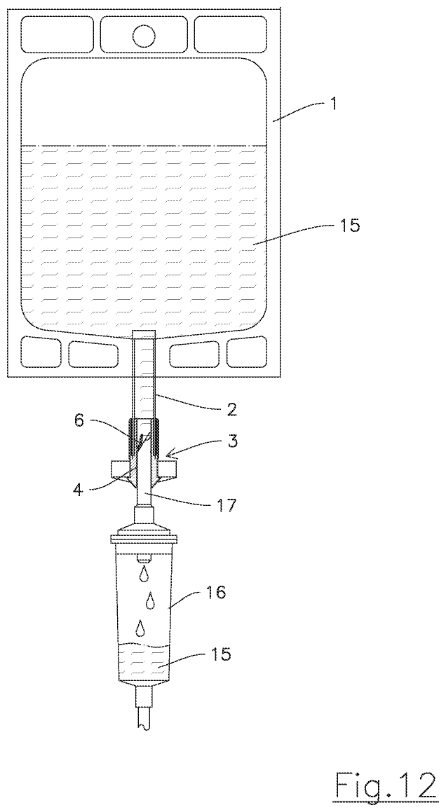

The connection of the infusion set or other dispensing device is carried out after detaching the closing part 8 at the frangible weakened section 9, so that the piercing terminal 17 can be introduced into the axial hole 5 of the main body 4 of connector 3 (FIG. 11) up to pierce the closing membrane 6 (FIG. 12). The liquid solution 15 can thus flow drop by drop from the bag 1 to the infusion set 16.

It is apparent that the coupling to coaxial tubes described for the dispensing connector 3 of the previous figures can be used for any other type of connector which is associable with a liquid solution bag such as that shown in the drawings. The protection deriving from the present application is thus to be considered as extended to connectors of any type which include a coupling element as defined in the following claims.

1. A connector applicable to a bag containing liquid solutions for medical or nutritional use, comprising a coupling part for coupling to an end of a small tube projecting from the bag, the coupling part containing an axial hole closed by a pierceable inner membrane, and a closing part placed to close the end of the axial hole and detachable from the coupling part, wherein the coupling part comprises two coaxial tubes which define an annular inner space therebetween, into which an end of the small tube is insertable by axial pressure, wherein the annular inner space extends axially with a constant cross-section, and the coupling part and the closing part form a single body provided with a frangible weakened section for the detachment of the closing part from the coupling part.

2. The connector according to claim 1, wherein the single body comprises gripping fins which extend outwards from the coupling part and the closing part, respectively, and are close to the weakened section of the single body.
